(19) United States
(12) Patent Application Publication
(10) Pub. No.: US 2002/0037855 A1
(43) Pub. Date: Mar. 28, 2002

(54) Stabilized medicament containing cysteinyl derivatives

(76) Inventor: Fritz Stanislaus, Muenchen (DE)

Correspondence Address: HELLER EHRMAN WHITE & MCAULIFFE LLP, 275 MIDDLEFIELD ROAD, MENLO PARK, CA, 94025-3506, US

(21) Appl. No.: 09/816,769

(22) Filed: Mar. 22, 2001

Related U.S. Application Data
(63) Continuation of application No. 09/403,160, filed on May 5, 2000, now abandoned.
Application No. 09/403,160 is a 371 of international application No. PCT/EP97/01941, filed on Apr. 18, 1997.

(51) Int. Cl. A61K 38/06; A61K 31/7048; A61K 31/60; A61K 31/404; A61K 31/352; A61K 31/355; A61K 31/375

(52) U.S. Cl. 514/18; 514/27; 514/162; 514/456; 514/458; 514/474; 514/569; 514/419; 514/420; 514/725

(57) ABSTRACT

The invention relates to a stabilized medicament with an amount of active ingredients containing cysteine groups and NSAID compounds, wherein a stabilization of the combination, especially the active ingredients containing the cysteine group, can be conducted with a mixture of at least three anti-oxidative components. The therapeutic and prophylactic use of this medicament stabilized in this manner lies in the field of the prevention and therapy of inflammatory diseases among the fields of medical indications.

The invention relates to medicaments with an amount of cysteine derivatives having an improved stability.

Medical substances with a cysteine group represent important members of the pharmacopoeia. N-acetylcysteine, which has a mucolytic effect in various diseases of the lung and bronchia and is therefore frequently prescribed as a secrolytic agent for these diseases, for example colds and inflamed bronchia and certain asthmatic states, is among their most important compounds. Aside from this, other cysteine derivatives, such as S-(carboxymethyl)-cysteine or N-acetyl-3-[2-benzoylpropyl)]-thioalanine (bencisteine) have taken on a certain importance as secrolytic agents for the above mentioned indications.

A further important group of therapeutically employed cysteine derivatives is represented above all by the so-called detoxification agents or detoxicants employed as physiological detoxicants or as liver protective agents as well. Among these are, for example, γ-L-glutamyl-L-cysteinyl-glycine (glutathione) which is also applied as an anabolic agent among others or L-β-β′-dithiodialanine (dithiobisalanine, dicysteine) which can be particularly prescribed against damage due to lack of protein, diseases of the liver parenchyma, pregnancy toxicities or furunculosis.

However, it has been determined that the acetylated cysteine is distinguished in the body by increased pharmacokinetic stability as compared to the starting substance cysteine. This is traced to the fact that the active ingredient taken up in an oral manner is not only present as a free substance but, in addition to this, is also reversibly bound to proteins over disulfide bridges or is tightly incorporated into proteins. The disulfide bridges arising between N-acetylcysteine and the proteins are labile such that the re-releasable active ingredient can exhibit its activity in the organism but cause the active ingredient to be protected. However, these active ingredients are not present in their physiological protected form in the medicaments so that they are exposed to considerably negative influences, for example through moisture, heat, oxygen or atmospheric oxidatively aggressive agents in connection with ozone, for example singlet oxygen, oxygen radicals, nitrogen oxides and other environmental toxic agents, even during storage of the medicaments containing them in the form of granulates, compressives, solutions etc.

Although the above mentioned dicysteine already has the mentioned disulfide bridge, this is also sensitive to the environmental influences and damaging storage effects discussed above. This is especially true with respect to light or illumination, heat or acids as is the extremely disadvantageous case with the popular intake of preparations of this type in teas or as effervescent formulations for the treatment of the above mentioned indications. This is especially true when the effervescent powder and/or effervescent granulate and/or effervescent tablets or granulate, tabs or other instant formulations for ingestion are to be dissolved in water, hot tee or in fruit juices according to the suggestions of the packaging instructions in the medicament.

Thus, it was determined in EP-A-0 349 797 that N-acetylcysteine formulations are relatively unstable with the ingestion with tea such that a considerable degradation of the active ingredient is to be noted with longer standing of the formulation. This problem is solved according to this publication by the addition of ascorbic acid and/or ascorbate, wherein the constituent amounts of ascorbic acid to active ingredient are preferably 5 to 100 parts and more preferably 10 to 50 parts ascorbic acid to 100 parts. In this connection, individual doses of 25, 50 and 100 mg were achieved according to the comparative graphs.

It has be been found in practice that the stabilization measures proposed in the above publication EP-A-0 349 797 are not sufficient in many cases for the following reasons: the pH value is shifted into the acidic range with increasing amounts of ascorbic acid such that certain oxidative influences arise more frequently; in addition, the acidic taste of the formulation can be unacceptable in an uncomfortable manner for a number of patients especially during the ingestion with fruit tees, fruit juices, etc. whereby, on the other hand, the stability of compounds, for example dicysteine mentioned above, is particularly negatively influenced in the acidic milieu; additionally, more current research has found that the daily vitamin intake can take on degrees undesired under physiological aspects as a result of partially uncontrolled enrichment of frequently ingested foods, for example, with fruit juices, ready-to-eat foods, fruit teas, etc.. This is especially true for ascorbic acid whose amount in the foods mentioned can even widely exceed the increased daily need of ill people, in other words, can widely exceed the physiologically meaningful supply, as a result of artificial additives. In the same way, this is also true for vitamins or their precursors, for example, carotinoids or vitamin E, which are very widely distributed in multiply uncontrolled manners whether they are prescribed by a doctor to prevent a vitamin deficiency or for therapeutic purposes or over-the-counter as vitamin preparations or by ingestion of fruit juices, vegetable juices or other foods fortified in vitamins.

It is also known that medicaments, especially those which in contrast to vaccines do not require an uninterrupted chain of cooling, can be subjected to massive losses of active ingredient even after relatively short storage periods by transport and/or storage in hot summer months even in cooler countries such as those in the more northern latitudes, not to mention Mediterranean countries or the tropics. Although well-contrived foil coatings have been developed by matured packaging technology which even make long storage times of medicaments possible at high temperatures and moisture values, these require complex machinery and expensive raw materials for production of the foils and their partially multilayered coating but also for the sealing of the finished dosage units. The spatial requirement is considerable as a result of the dimensions of the blister packaging such that the transport alone can turn out to be very uneconomical as a result of the large package volume. Ecological difficulties represent much larger problems, for example with the complicated disposal of PVC or fluorine-containing polymers, such that the use of packages of this type improved with respect to the storage stability has become undesirable.

Therefore, the applicant was faced with the problem to not only improve the stability of formulations containing cysteinyl groups with respect to the discussed problems on the one hand, but to simultaneously diminish undesirable, high stabilizing amounts of ascorbic acid and/or ascorbate.

The object to improve the stabilization in multiple respects was of even greater priority as the applicant found out that cysteine derivatives are suitable for the combined use in the administration of non-steroid inflammation inhibitors (NSAID) and analgesic agents, such as Diclofenac for example. Furthermore, the applicant determined that the inflammation-inhibiting effect of NSAID active ingredients of this type can be amplified in a super-additive, i.e. synergistic, manner by the combined administration and/or by suitable combination medicaments together with the cysteine derivatives, particularly glutathione or N-acetylcysteine, for the treatment of inflammatory diseases of the bronchia for example. As a result, not only can the damaging side-effects of these pharmaceuticals be reduced in an unexpected manner, but their therapeutic effectiveness can be surprisingly increased in inflammatory diseases for example. Therefore, to achieve the maximal possible synergistic effects, it is desirable to bring about the complete effect of cysteine derivatives, such as γ-L-glutamyl-L-cystenylglycine for example, which is only possible with suitable, further stabilizing additives and/or measures with amounts of ascorbic acid that are as small as possible.

This problem was solved in a completely unexpected manner by providing a stabilized medicament with an amount of cysteinyl or dicysteinyl compounds, particularly N-acetylcysteine, against bronchia and lung disease, and/or glutathione and/or γ-L-glutamyl-L-cystenylglycine, optionally in combination with a non-steroid anti-phlogistic agent (NSAID) and/or non-steroid analgesic agent, particularly Diclofenac, for the suppression of organ particularly liver, damage and inflammation damage as sole active ingredient(s), wherein this contains a stabilizer mixture comprising at least 3 components of the series

a) ascorbic acid (vitamin C) or salts or esters thereof,

b) one or more tocopherols (vitamin E),

c) one or more carotinoids and/or vitamin A,

d) one or more natural or synthetic flavonoids, bioflavonoids, flavanols or catechins including anthocyanenes and derivatives such as glycosides

aside from pharmaceutically acceptable adjuvants, additives and/or carrier agents for parenteral, per oral, topical or rectal administration forms if required.

As non-steroid antiphlogistic agents and/or analgesic agents, the stabilized medicament can contain one or more compounds selected from group consisting of paraaminophenols, particularly paracetamol, salicylates, particularly acetylsalicylic acid, diflunisal or choline salicylate, acetic acid derivatives such as indometacin or acemetacin, propionic acid derivatives, particularly ibuprofen, ketoprofen or naproxen, indole derivatives, particularly sulindac, oxicam derivatives such as priroxicam, fenamate derivatives such as mefenaminic acid or pyrzole derivatives, particularly phenylbutazone, phenazone, propylphenazone or metamizole, as well as their optionally existing pharmacologically active enantomers or other optical isomers.

In the stabilized medicament according to the invention, the component b) can be selected from one or more compounds as well as their derivatives of natural or synthetic origin from the series α-, β-, γ-, δ- and ε-tocopherol as well as all-rac-α-tocopherol, tocol, α-tocopherolhydroquinone, α-tocopherolquinone, their derivatives such as acetates, succinates, nicotinates or poly(oxyethylene)succinates, ubiquinones, (boviquinones), (plastoquinones) or menaquinones.

A further embodiment according to the invention is that the component c) is selected from one or more compounds, as well as their derivatives of natural or synthetic origin, from the series cathaxanthin, rhodoxanthin, capsorubin, zeaxanthin, α-, β-, γ-, δ- and/or ζ-carotine, lycopin, capsanthin, cryptoxanthin, crocetin, lutein, decapren-β-carotine, dodecapren-β-carotine, astaxanthin, violaxanthin or bixin.

In a preferred embodiment of the medicament according to the invention, the component d) is selected from one or more compounds, as well as their derivatives of natural or synthetic origin, from the series of flavonoids and/or bioflavonoids such as chrysin, apigenin, fisetin, kaempferol, luteolin, galangin, gossypetin, morin, myricetin, naringin, quercetin, robinetin, anthocyanins, rutin, hesperidine, taxifolin, catechinic acid, epicatechol, epicatechol gallate, gallocatechol, epigallocatechol gallate, tangeretin, eriodictyol, naringenin, rutin, troxerutin, quercetinrutin, esculin, esculetin, skimmin, umbelliferon, corresponding other glycosides such as esculoside or anthocyanosides, rutosides such as tri(hydroxyethyl)rutoside, ruscogenins, O-(β-hydroxyethyl)-rutocides as well as natural extracts from citrus fruits, Myrtillum species, Melilotus species, Hypericum species, or other plant extracts supplying bioflavonoids.

According to a further embodiment of the invention, the medicament contains an additional stabilizer agent according to component d) in the form of one or more compounds, as well as their derivatives of natural or synthetic origin, from the series of polyphenols such as caffeic acid, caffeic acid amide, ethoxyquin, carosinic acid, carnosol, as well as derivatives of these compounds, extracts from thea species, rosemary species or other plants supplying natural phenol compounds of these types.

In a preferred manner, the stabilized medicament according to the invention can have an amount of trace elements, above all selenium, for example as the sodium selenite or sodium selenate, which themselves not only exert an additional stabilizing effect on the medicine, but also exert an amplified therapeutic and prophylactic effect in a super-additive manner in the indications described above, for example in inflammation or with diseases connected with toxic effects of free radicals. The individual doses in mg are, for example, approximately 0.01, 0.02, 0.05, 0.1; the daily doses in mg are, for example, approximately 0.1, 0.2, 0.24, 0.4, 0.5 up to a maximum 0.8 or 1.0 mg, the last dose corresponding to 1000.0 μg.

Furthermore, a preferred embodiment of the stabilized medicament is that the liquid aqueous or lipid-containing formulations can contain, as solubilizers, a solubility promoter from the series of the phospholipids, for example lecithins or polyoxyethylene glycol fatty acid esters, in the form of the monolaurate, monostearate, monooleate, triricinoleate or trihydroxystearate (cremophor), tegin types, aside from optional organic, pharmaceutically acceptable solvents such as alcohols in the form of ethanol, isopropanol, butanol. The active ingredients defined above or several of the active ingredients defined above, particularly N-acetylcysteine or the NSAID medicament, can be present in liposomal, finely dispersed or target form.

According to a particularly preferred embodiment of the invention, the quantitative relation of (i) cysteinyl and/or dicysteinyl derivatives, particularly N-acetylcysteine and/or (ii) glutathione (γ-L-glutamyl-L-cysteinyl-glycine): (iii) one or more non-steroid antiphlogistic agents/analgesic agents (NSAID): (iv) stabilizer mixture is 0 or 0.05-2.0 percent by weight (i) and/or 0 or 0.05-2.0 percent by weight (ii): 0-2.0 or 4 or 5 percent by weight (iii) : 0.02 or 0.05-1.0 percent by weight (iii).

It has been determined in an unexpected manner that the stabilizing effect of ascorbic acid in comparison to the amount necessary according to EP-A-0 349 797 is increased in a super-additive manner. Thus, the amount of 10 mg that has a stabilizing effect according to the diagram depicted in this publication is sufficient to achieve or even exceed the best stability values apparent from that diagram. This means that the stabilizing effect can be adjusted by the selection of the other stabilizer components in such a manner that the ascorbic acid amount can be regulated to the desired low level, for example down to 2.3 or 5 mg per dosage unit, whereby the total stability can be increased even further.

Within the framework of the above quantitative relation with respect to the compounds (i), (ii) and (iii), 50 mg, 100 mg, 125 mg, 150 mg, 200 mg, 50 mg, 300 mg, 400 mg, 500 mg, 600 mg, 800 mg or 1000 mg are considered in each case for example. This applies in a typical manner for the above mentioned cysteinyl as well as NSAID compounds, particularly N-acetylcysteine and Diclofenac, but also for the analogously structured and/or bioequivalently effective medical substances. The respective daily doses per patient and individual active ingredient and/or the above mentioned active ingredient groups can each be 300 mg, 500 mg, 1000 mg, 1500 mg, 2000 mg, 2500 mg and up to 3000 mg; in extreme cases, up to 4000 mg or 5000 mg. Correspondingly, the constituent amounts of the stabilizer mixture (iv) in the medicament can be up to 500 mg per dosage unit and/or individual dose as given above for the active ingredients, but can also be lower than 50 mg; for example, the amount of (iv) can only be 5 mg, 10 mg, 20 mg, 25 mg or 30 mg for example. The respective amount of ascorbic acid per dosage unit does not need to exceed 50 mg. The constitutive amounts of the other stabilizers b), and/or c) and/or d) can be selected within the given framework depending on the desired purpose, for example 10 mg and/or percent by weight a) +30 mg and/or percent by weight b) +60 mg and/or percent by weight d) or 5 mg and/or percent by weight a) +20 mg and/or percent by weight c) +75 mg and/or percent by weight d) or 5 mg and/or percent by weight a) +15 mg and/or percent by weight a) +25 mg and/or percent by weight b) +60 mg and/or percent by weight c).

The quantitative relation within the stabilizer mixture as well as the active ingredients can be freely varied such that the above amounts of the stabilizer components and active ingredient dosages are merely to be understood as examples. The amount of stabilizer can be reduced depending on the active ingredient dose, i.e. halved, divided into thirds, quartered or also multiplied by doubling or trebling etc. A stabilizer mixture of 4 components can be composed of 12 mg and/or percent by weight a) +24 mg and/or percent by weight b) +24 mg and/or percent by weight c) +40 mg and/or percent by weight d) per dosage unit. Within the given quantitative relations expressed as parts by weight, the respective individual doses of the components of the stabilizer mixture can be in milligram, for example 1, 2, 3, 4, 5, 6, 10, 12, 15, 20, 25, 30, 35, 40, 50, 60, 70, 75, 80, 100 or 120 mg. However, they are preferably not more than 150 or 200 mg. In this connection, these parts by weight can also take on every even or odd value found between them. The respective suitable quantitative relation of the components among themselves, for example the ascorbic acid compound to flavonoids, carotinoids, tocopherols and/or polyphenols and/or the corresponding extracts mentioned above, can be adjusted by customary stabilizer experiments such as tests under intensified conditions, for example at increased humidity and temperature, in a relatively simple and known manner depending on the employed, optionally combined, active ingredients.

The active ingredients from the group of cysteinyl compounds can be processed to stabilized medicaments separately from medicines and/or dosage units with an amount of the NSAID medicaments, i.e. more precisely stated, oral solid medicaments, for example tablets with one or more cysteinyl compounds and vials with an amount of one or more NSAID antiphlogistic agents, can be separately arranged in a medicine package next to each other. In other words, in the case of a combination of the two active ingredient groups, an agent containing the cysteinyl compound on the one hand and the medicament containing a non-steroid antiphlogistic agent on the other hand, can be applied independently of each other, for example at the same time or shifted in time: for example, N-acetylcysteine 3-4 times daily and the NSAID, such as Diclofenac, 1-2 times daily. The therapy and dosage scheme to be adhered to thereby is to be determined by the doctor depending individually on the patient, his clinical picture and physiological status and fluctuates within the framework of the generally known administration schemes or within the individual dispensation and prescription practice of the physician.

In the case of the combination of one or more cysteinyl-containing compounds with the NSAID antiphlogistic agent/analgesic agent, suitable medicaments are also considered with an amount of compounds from the two active ingredient groups and/or separately present medicine packages or medicine packages to be administered for the combination that are not provided with the above stabilizer mixture in a particular manner. The dosages of the active ingredients considered for this correspond to those named above. A reduced stability and a decreased activity in the different medical indications named below, especially those in connection with the suppression of the formation of free radicals or their neutralization and/or detoxification/decontamination is to be accepted in the case of the active ingredient combinations without stabilizer additives. The above named dosages, dose units and quantitative relations apply for the active ingredient combinations of the compound groups (i) plus optionally (ii) plus optionally (iii) in the same manner.

The medicaments stabilized in this manner can be present in the form of optionally coated, gastric juice resistant and/or retarded capsules, compressives, sublingual or chewable tablets, chewing gum, lozenges, effervescent formulations, tabs, granulates, pellets, microcapules, dry juices, powders, injection and infusion preparations, suppositories, rectal or vaginal capsules, drops, suspensions, solutions, dosing aerosols, nebulizers, atomizers, sprays, liposome formulations, transdermal formulations, plasters, pastes, emulsions, creams or gels, otologic agents, ophthalmologic agents ovula or instillation solutions.

The medicines according to the invention can be particularly in the form of granulates, dry juice, pellets or liposome formulations, an effervescent tablet, effervescent granulate or tabs, whereby the active ingredient or the active ingredients are processed as (i) cysteinyl compound, particularly N-acetylcysteine, in individual doses of 10 mg, 20 mg, 25 mg, 50 mg, 100 mg or 200 mg and/or the respective 2-, 3-, 4- or 5-fold amount thereof together with the stabilizer mixture of at least 3 or 4 of the components a), b), c) and d) from the series ascorbic acid compound, vitamin A (carotinoids), vitamin E (tocopherols) and/or one or more flavonoids such as rutin, esculin, catechinic acid, plant polyphenols such as caffeic acid and/or one or more extract(s) from citrus fruits containing bioflavonoids in individual dosages of 2.0 mg, 2.5 mg, 5.0 mg, 10 mg or 20 mg each and/or the respective 2-, 3-, 4-, 5- or 10-fold amount thereof, optionally in combination with glutathione (ii) in individual dosages of 25 mg, 50 mg, 100 mg, 200 mg or 250 mg and/or the respective 2-, 3-, or 4-fold amount thereof and optionally further in combination with (iii) NSAID compounds, particularly Diclofenac, each in an amount of 25 mg, 50 mg, 100 mg or 200 mg and/or the respective 2-, 3-, 4- or 5-10-fold amount thereof together with customary tableting and granulating adjuvants as well as organic acids developing carbon dioxide in effervescent formulations such as citric or tartaric acid and carbonates such as monosodium carbonate, granulation adjuvants, flavoring agents, sweetening agents such as saccharin and/or sugars such as mannitol, sorbitol and customary carrier agents if necessary.

During the administration of the especially stabilized medicaments according to the invention, it was determined that these pharmaceutical agents demonstrate a number of physiologically important valuable properties and effects, for example, the reduction of muscle damage during physiological oxidative stress, the arrest of diabetic vessel damage, the decrease of oxidative stress in smokers, the decrease of (late) damage in the administration of cytostatic agents, especially as triggered by their long-term application, protective effectiveness against skin damage by excess UV radiation or other damaging radiation or weather influences, especially those caused by oxidative effects of air contaminants, for example, by ozone, nitrous oxide, oxygen radicals, singlet oxygen and other aggressive radicals.

A favorable reduction of inflammation mediator synthesis and formation of thromboxane as well as the cell proliferation promoting factor PDGF (platelet-derived growth factor) in connection with the supply of ω-3 fatty acids (ωb-3-PUFA),is a further favorable effect of the especially stabilized medicament containing a cysteinyl compound according to the invention with the increased oxidative status in the organism associated with this, be it when this is taken with food or be it in the form of a medicinal supplement agent. This is also true for a lowering of the increased fibrinogen concentrations arising in this case such that the effect of the NSAID is promoted in an unexpected manner by the combination according to the invention, especially with glutathione or physiological precursors thereof and/or its esters as cysteinyl compounds.

The lastly mentioned cysteine derivatives and cysteinyl compounds comparable to these play a role in the anti-oxidative protection mechanisms in inflammation processes caused by pulmonary and bronchial diseases, excessive tobacco smoking, ozone load and toxic radicals present in the air and noxins which are also associated with increased inflammation mediator, inflammation cell and free radical production. It was determined according to the invention that the sole administration of the above mentioned cysteinyl compounds together with the anti-oxidative agent mixture already brings about inflammation inhibiting effects without further active ingredients, i.e. also without NSAID, and further, that the physiological damage caused by inflammations is decreased in a super-additive manner in each case.

In this connection, these advantageous effects are especially seen in different organs such as the lung, kidney, liver or heart, these organs being subjected to particularly strong loads through correspondingly disadvantageous environmental influences such as air, water and food pollutants which the organism is often no longer able to overcome with its own power such that numerous types of partially irreparable organ damage and chronic diseases are a daily occurrence. This is even more so since the content of glutathione, for example, naturally present in numerous foods, for example vegetables or fruits, can be greatly decreased through storage of processing. Therefore, the medicament cysteinyl-containing medicaments according to the invention are not only suitable for the super-additive substitution of cysteinyl-containing substances, but are also very excellently suitable for the prevention and therapy of stress-related, preferably inflammatory, diseases of the above mentioned organs such as pneumonia, hepatitis, nephritis, arteriosclerosis, vein diseases, arthritis of different genesis or immune and/or autoimmune diseases, transplantation rejections, especially of the inflammatory type, late diabetic consequences on vessels, kidney and retina, etc., radiation damage, perfusion or reperfusion damage, and also particularly for the compensation of subsequent damage caused by medicaments, for example, after antibiotic agent, analgesic agent or cytostatic agent administration etc. The stabilized, cysteinyl-containing medicaments according to the invention are also suitable for the detoxification after the effects of toxicity of the most varied noxins, for example, of cytotoxic lipid peroxidation products among others. Therefore, the present stabilized formulations according to the invention also represent an excellent buffer system for the redox state of the cell as a result of the present SH and disulfide groups such that the effects described above are particularly strongly effective. Preferably, the especially stabilized medicaments containing cysteinyl compounds according to the invention can be employed in an advantageous manner in connection with an inhibition or prevention of apoptosis, for example, following a medical normalization of the coronary blood flow, whereby the tissue bordering an infarct region is increasingly inundated with damaging free radicals as a result of the increase of the blood flow. This toxic effect can be therapeutically and prophylactically influenced in a favorable manner by the medicaments according to the invention. The cellular and/or tissue mechanisms of apoptosis processes are described in detail in “Apoptosis and Programmed Cell Death in Immunity ”, J. J. Cohen, R. C. Duke, V. A. Fadok and K. S. Sellins, Annual Review of Immunology, Vol. 10, (1992), pages 267-293 or “Apoptosis: A Basic Biological Phenomenon with Wide-Ranging Implications, British Journal of Cancer, Vol. 26, (1972), pages 239-257 for example.

The effect of the stabilized medicaments with the anti-oxidant agent mixture according to the invention can be further increased by the production of liposome formulations, wherein fine particles of the cysteinyl compounds and/or NSAID active ingredients are coated with a membrane in a customary manner.

It could also be determined that the cysteinyl-containing pharmaceutical agents according to the invention are suitable in the form of liquid formulations, for example solutions with an amount of the anti-oxidative agent mixture defined above, for the extended preservation or the perfusion of organs for autogenic or allogenic transplantation of heart, liver, kidney, lung, vessels such as veins, pancreas or islet cells for example.

As a result of its detoxifying effect on carcinogenic agents, a further medical indication of the especially stabilized medicaments according to the invention is the prevention of bronchial carcinomas and is applicable to other carcinomas. As mentioned above, the medicaments according to the invention can also be applied in combination with a cytostatic agent in the therapy of those particular neoplastic growths. Thus, this results in not only a suppression in the formation of or the effect of carcinogenic substances already present, but also, at the same time, an advantageous detoxifying effect of the cancerostatic agents, immunosuppressive agents and cytostatic agents partially afflicted with strong side effects.

As a result of the particular properties of the medicament according to the invention, this exerts a prophylactic and therapeutic effect without an additional amount of NSAID in inflammatory diseases such as rheumatic diseases, for example arthritic or vein diseases, hemorrhoid symptoms, thrombopathies or thromoses such that use with patients sensitive to customary anti-rheumatic agents leads to an unexpected shortening of the course of the disease. As discussed above, this is a further favorable advantageous property of the present medicament especially with an amount of the stabilizer mixture.

With respect to the state of the art on liposomal formulations, for example of N-acetylcysteinyl liposomes, U.S. Pat. No. 4 895 719 (=EP-A-O 223 831) and the publication WO-A-9 116 882 are referred to and their methods of production are incorporated herein.

### Therapeutic Administration Forms

The production of medicaments with an amount of the above mentioned compounds occurs in the customary manner by means of common pharmaceutical technology methods. For this, the active ingredients as such or in the form of their salts are processed together with suitable, pharmaceutically acceptable adjuvants and carriers to medicinal forms suitable for the various indications and types of application. Thereby, the medicaments can be produced in such a manner that the respective desired release rate is obtained, for example a quick flooding and/or a sustained or depot effect. In this connection, the modulation of the controlled release over longer or shorter times and at different locations of the intestinal tract such as the stomach and various intestine sections can occur by mixing different releasing pellet types, polymeric coatings, pH dependent substances, core/jacket tablets, mult-layered tablets, polymeric and/or lipid matrix and/or builder substances or combined retarding measures.

The separate processing of the active ingredients (i) and/or (ii) with the stabilizer on the one hand and one or more of the combined active ingredients (iii) on the other hand is preferred in the case of active ingredient combinations. This means the stability of the active ingredients (i) and (ii) can be further increased by the separate production of, for example, a granulate, of pellets or coated powders, liposomes, etc. containing (i) and/or (ii), the cysteinyl compounds, on the one hand and a second granulate containing (iii), NSAID, on the other hand.

Injections can also be administered aside from per oral administrated medicaments. These are prepared either in the form of vials or also as so-called ready-to-use injection preparations, for example as ready-to-use syringes or single use syringes in addition to perforation bottles for multiple withdrawals. Administration of the injection preparations or infusion solutions can occur in the form of subcutaneous (s.c.), intramuscular (i.m.), intravenous (i.v.) or intracutaneous (i.c.) application. The respective suitable injection forms can especially be produced as solutions, crystal suspensions, nanoparticular or colloid-disperse systems, such as for example, hydrosols.

The injectable formulations can also be produced as concentrates which can be adjusted with aqueous isotonic dilution agents to the desired active ingredient dosage. Furthermore, they can also be produced as powders, such as for example lyophilisates, which are then preferably dissolved or dispersed immediately before application with suitable diluents. The infusions can also be formulated in the form of isotonic solutions, fat emulsions, liposome formulations, microemulsions and liquids based on mixed micells, for example, based on phospholipids. As with injection preparations, infusion formulations can also be prepared in the form of concentrates to dilute. The injectable formulations can also be applied in the form of continuous infusions as in stationary as well as in out-patient therapy, for example in the form of mini-pumps.

Albumin, plasma expanders, surface active compounds, organic solvents, pH influencing compounds, complex forming compounds or polymeric compounds can be added to the parenteral medicinal forms, especially as substances for influencing the adsorption of the active ingredients to protein or polymers or also with the aim of decreasing the adsorption of the active ingredient to materials such as injection instruments or packaging materials, for example plastic or glass.

The active ingredients can be bound to nanoparticles in the preparations for parenteral use, for example on finely dispersed particles based on poly(meth)acrylates, polyacetates, polyglycolates, polyamino acids or polyether urethanes. The parenteral formulations can also be modified as depot preparations, for example based on the multiple unit principle where the active ingredients are incorporated in a most finely distributed and/or dispersed, suspended form or as crystal suspensions, or on the single unit principle where the active ingredient is enclosed in a medicinal form, for example, a tablet or a seed which is subsequently implanted. Often, these implantations or depot medicaments in single unit and multiple unit medicinal forms consist of so-called biodegradable polymers, such as for example, polyether urethanes of lactic and glycolic acid, polyether urethanes, polyamino acids, poly(meth)acrylates or polysaccharides.

Sterilized water, pH value influencing substances, such as for example organic and inorganic acids or bases as well as their salts, buffer substances for setting the pH value, agents for isotonicity, such as for example sodium chloride, monosodium carbonate, glucose and fructose, tensides and/or surface active substances and emulsifiers, such as for example, partial fatty acid esters of polyoxyethylene sorbitan (Tween®) or for example fatty acid esters of polyoxyethylene (Cremophor®), fatty oils such as for example peanut oil, soybean oil and castor oil, synthetic fatty acid esters, such as for example ethyl oleate, isopropyl myristate and neutral oil (Miglyol®) as well as polymer adjuvants such as for example gelatin, dextran, polyvinylpyrrolidone, organic solvent additives which increase solubility, such as for example propylene glycol, ethanol, N,N-dimethylacetamide, propylene glycol or complex forming compounds such as for example citrates and urea, preservatives, such as for example hydroxypropyl benzoate and hydroxymethyl benzoate, benzyl alcohol, anti-oxidants, such as for example sodium sulfite and stabilizers, such as for example EDTA, are suitable as adjuvants and carriers in the production of preparations for parenteral use.

In suspensions, addition of thickening agents to prevent the settling of the active ingredients from tensides and peptizers, to secure the ability of the sediment to be shaken, or complex formers, such as EDTA, ensues. This can also be achieved with the various polymeric agent complexes, for example with polyethylene glycols, polystyrol, carboxymethylcellulose, Pluronics® or polyethylene glycol sorbitan fatty acid esters. The active ingredient can also be incorporated in liquid formulations in the form of inclusion compounds, for example with cyclodextrins. As further adjuvants, dispersion agents are also suitable. For production of lyophilisates, builders are also used, such as for example mannitol, dextran, saccharose, human albumin, lactose, PVP or gelatin varieties.

As long as the active ingredients are not incorporated in the liquid medicinal formulations in the form of a base, they are used in the form of their acid addition salts, hydrates or solvates in the preparations for parenteral use.

A further systemic application form of importance is per oral administration as tablets, hard or soft gelatin capsules, coated tablets, powders, pellets, microcapsules, oblong compressives, granules, chewable tablets, lozenges, gums or sachets. These solid, per oral administration forms can also be prepared as sustained action and/or depot systems. Among these are medicaments with an amount of one or more micronized active ingredients, diffusions and erosion forms based on matrices, for example by using fats, wax-like and/or polymeric compounds, or so-called reservoir systems. As a retarding agent and/or agent for controlled release, film or matrix forming substances, such as for example ethylcellulose, hydroxypropylmethylcellulose, poly(meth)acrylate derivatives (for example Eudragit®), hydroxypropylmethylcellulose phthalate are suitable in organic solutions as well as in the form of aqueous dispersions. In this connection, so-called bio-adhesive preparations are also to be named in which the increased retention time in the body is achieved by intensive contact with the mucus membranes of the body. An example of a bio-adhesive polymer is the group of Carbomers®.

For sublingual application, compressives, such as for example non-disintegrating tablets in oblong form of a suitable size with a slow release of active ingredient, are especially suitable. For purposes of a targeted release of active ingredients in the various sections of the gastrointestinal tract, mixtures of pellets which release at the various places are employable, for example mixtures of gastric fluid soluble and small intestine soluble and/or gastric fluid resistant and large intestine soluble pellets.

The same goal of releasing at various sections of the gastrointestinal tract can also be conceived by suitably produced laminated tablets with a core, whereby the coating of the agent is quickly released in gastric fluid and the core of the agent is slowly released in the small intestine milieu. The goal of controlled release at various sections of the gastrointestinal tract can also be attained by multilayer tablets. The pellet mixtures with differentially released agent can be filled into hard gelatin capsules.

Anti-stick and lubricant and separating agents, dispersion agents such as flame dispersed silicone dioxide, disintegrants, such as various starch types, PVP, cellulose esters as granulating or retarding agents, such as for example wax-like and/or polymeric compounds on the basis of Eudragit®, cellulose or Cremophor® are used as a further adjuvants for the production of compressives, such as for example tablets or hard and soft gelatin capsules as well as coated tablets and granulates.

Anti-oxidants, sweetening agents, such as for example saccharose, xylitol or mannitol, masking flavors, aromatics, preservatives, colorants, buffer substances, direct tableting agents, such as for example microcrystalline cellulose, starch and starch hydrolysates (for example Celutab®), lactose, polyethylene glycols, polyvinylpyrrolidone and dicalcium phosphate, lubricants, fillers, such as lactose or starch, binding agents in the form of lactose, starch varieties, such as for example wheat or corn and/or rice starch, cellulose derivatives, for example methylcellulose, hydroxypropylcellulose or silica, talcum powder, stearates, such as for example magnesium stearate, aluminum stearate, calcium stearate, talc, siliconized talc, stearic acid, acetyl alcohol and hydrated fats are used.

In this connection, oral therapeutic systems constructed especially on osmotic principles, such as for example GIT (gastrointestinal therapeutic system) or OROS (oral osmotic system), are also to be mentioned.

Effervescent tablets, effervescent granulates and tabs, both of which represent immediately drinkable instant medicinal forms which are quickly dissolved or suspended in water, are among the per oral administratable compressives. Systems for the development of gaseous CO₂ formed in aqueous milieu in situ such as organic acids, for example citric acid and carbonates, are employed among others as granulation adjuvants for the production of the mentioned effervescent forms.

Among the per oral administratable forms are also solutions, for example drops, juices and suspensions, which can be produced according to the above given method, and can still contain preservatives for increasing stability and optionally aromatics for reasons of easier intake, and colorants for better differentiation as well as antioxidants and/or vitamins and sweeteners such as sugar or artificial sweetening agents. This is also true for dry juices which are formulated with water before ingestion. Ion exchange resins in combination with one or more active ingredients are also to be mentioned for the production of liquid ingestable forms.

A special release form consists in the preparation of so-called floating medicinal forms, for example based on tablets or pellets which develop gas after contact with body fluids and therefore float on the surface of the gastric fluid. Furthermore, so-called electronically controlled release systems can also be formulated by which active ingredient release can be selectively adjusted to individual needs.

A further group of systemic administration and also optionally topically effective medicinal forms are represented by rectally applicable medicaments. Among these are suppositories and enema formulations. The enema formulations can be prepared based on tablets with aqueous solvents for producing this administration form. Rectal capsules can also be made available based on gelatin or other carriers.

Hardened fat, such as for example Witepsol®, Massa Estarinum®, Novata®, coconut fat, glycerol-gelatin masses, glycerol-soap-gels and polyethylene glycols are suitable as suppository bases.

As topically, locally or regionally administration medicaments, the following are suitable as special formulations: vaginally or genitally applicable emulsions, creams, foam tablets, depot implants, ovular or transurethral administration installation solutions. For opthalmological application, highly sterile eye ointments, solutions and/or drops or creams and emulsions are suitable.

In the same manner, corresponding otological drops, ointments or creams can be designated for application to the ear or in the nose. For both of the above-mentioned applications, the administration of semi-solid formulations, such as for example gels based on Carbopols® or other polymer compounds such as for example polyvinylpyrolidone and cellulose derivatives is also possible.

For customary application to the skin or also to the mucus membrane, normal emulsions, gels, ointments, creams or mixed phase and/or amphiphilic emulsion systems (oil/water-water/oil mixed phase) as well as liposomes and transfersomes can be named. Sodium algenate as a gel builder for production of a suitable foundation or cellulose derivatives, such as for example guar or xanthene gum, inorganic gel builders, such as for example aluminum hydroxides or bentonites (so-called thixotropic gel builder), polyacrylic acid derivatives, such as for example Carbopol®, polyvinylpyrrolidone, microcrystalline cellulose or carboxymethylcellulose are suitable as adjuvants and/or carriers. Furthermore, amphiphilic low and high molecular weight compounds as well as phospholipids are suitable. The gels can be present either as hydrogels based on water or as hydrophobic organogels, for example based on mixtures of low and high molecular paraffin hydrocarbons and vaseline.

Pharmaceutical and/or dermatological formulations of this type are not only outstandingly suitable as medicaments for the above mentioned indications, but also as excellent dermal or mucosal protective layers or protecting screens against aggressive environmental influences.

Anionic, cationic or neutral tensides can be employed as emulsifiers, for example alkalized soaps, metal soaps, amine soaps, sulfanated or sulfonated compounds, cationic soaps, high fatty alcohols, partial fatty acid esters of sorbitan and polyoxyethylene sorbitan, for example lanette types, wool wax, lanolin, or other synthetic products for the production of oil/water and/or water/oil emulsions.

Hydrophilic organogels can be formulated, for example, on the basis of high molecular polyethylene glycols. These gel-like forms are washable. Vaseline, natural or synthetic waxes, fatty acids, fatty alcohols, fatty acid esters, for example as mono-, di-, or triglycerides, paraffin oil or vegetable oils, hardened castor oil or coconut oil, pig fat, synthetic fats, for example based on acrylic, caprinic, lauric and stearic acid, such as for example Softisan® or triglyceride mixtures such as Miglyol® are employed as lipids in the form of fat and/or oil and/or wax-like components for the production of ointments, creams or emulsions.

Osmotically effective acids and bases, such as for example hydrochloric acid, citric acid, sodium hydroxide solution, potassium hydroxide solution, monosodium carbonate, further buffer systems, such as for example citrate, phosphate, Tris-buffer or triethanolamine are used for adjusting the pH value.

Preservatives, for example such as methyl- or propyl benzoate (parabenes) or sorbic acid can be added for increasing stability.

Pastes, powders or solutions are to be mentioned as further topically applicable forms. Pastes often contain lipophilic and hydrophilic auxiliary agents with very high amounts of fatty matter as a consistency-giving base.

Powders or topically applicable powders can contain for example starch varieties such as wheat or rice starch, flame dispersed silicon dioxide or silica, which also serve as diluents, for increasing flowability as well as lubricity as well as for preventing agglomerates.

Nose drops or nose sprays serve as nasal application forms. In this connection, nebulizers or nose creams or ointments can come to use.

Furthermore, nose spray or dry powder formulations as well as controlled dosage aerosols are also suitable for systemic administration of the active ingredients.

These pressure and/or controlled dosage aerosols and dry powder formulations can be inhaled and/or insufflated. Administration forms of this type also certainly have importance for direct, regional application in the lung or bronchi and larynx. Thereby, the dry powder compositions can be formulated for example as active ingredient-soft pellets, as an active ingredient-pellet mixture with suitable carriers, such as for example lactose and/or glucose. For inhalation or insufflation, common applicators are suitable which are suitable for the treatment of the nose, mouth and/or pharynx. The active ingredients can also be applied by means of an ultrasonic nebulizing device. As a propellant gas for aerosol spray formulations and/or controlled dosage aerosols, tetrafluoroethane or HFC 134a and /or heptafluoropropane or HFC 227 are suitable, wherein non-fluorinated hydrocarbons or other propellants which are gaseous at normal pressure and room temperature, such as for example propane, butane or dimethyl ether can be preferred.

Instead of controlled dosage aerosols, propellant-free, manual pump systems can also be used.

The propellant gas aerosols can also suitably contain surface active adjuvants, such as for example isopropyl myristate, polyoxyethylene sorbitan fatty acid ester, sorbitan trioleate, lecithins or soya lecithin.

For regional application in situ, solutions for installation, for example for transurethral administration in bladder tumors or genital tumors, or for profusion in liver tumors or other organ carcinomas are suitable.

The respective suitable medicinal forms can be produced in accordance with the prescription regulations and procedures based on pharmaceutical-physical fundamentals as they are described for example in the following handbooks and are included in the present inventive subject-matter with respect to the production of the respective suitable medicaments:

Physical Pharmacy (A. N. Martin, J. Swarbrick, A. Cammarata), 2^{nd} Ed., Philadelphia, Pa., (1970), German edition: Physikalische Pharmazie, (1987), 3^{rd} edition, Stuttgart;

R. Voigt, M. Bornschein, Lehrbuch der pharmazeutischen Technologie, Verlag Chemie, Weinheim, (1984), 5^{th} edition;

P.H. List, Arzneiformenlehre, Wissenschaftliche Verlagsgesellschaft mbH, Stuttgart, (1985), 4^{th} edition;

H. Sucker, P. Fuchs, P. Speiser, Pharmazeutische Technologie, Georg Thieme Verlag, Stuttgart-New York, (1991), 2^{nd} edition;

A. T. Florence, D. Attwood, Physicochemical Principles of Pharmacy, The Maxmillan Press Ltd., Hongkong, (1981); Hager's Handbuch der Pharmazeutischen Praxis, 5^{th} edition;

L. A. Trissel, Handbook on injectable Drugs, American Society of Hospital Pharmacists, (1994), 8^{th} edition;

Y. W. Chien, Transdermal Controlled Systemic Medications, Marcel Dekker Inc., New York-Basel, (1987);

K. E. Avis, L. Lachmann, H. A. Liebermann, Pharmaceutical Dosage Forms: Parenteral Medications, Volume 2, Marcel Dekker Inc., New York-Basel (1986);

B. W. Müuller, Controlled Drug Delivery, Paperback APV, volume 17, Wissenschaftliche Verlagsgesellschaft mbH, Stuttgart, (1987);

H. Asch, D. Essig, P. C. Schmidt, Technologie von Salben, Suspensionen und Emulsionen, Wissenschaftliche Verlagsgesellschaft mbH, Stuttgart, (1984);

H. A. Liebermann, L. Lachmann, J. B. Schwartz, Pharmaceutical Dosage Forms: Tablets, Volume 1, Marcel Dekker Inc., New York, 2^{nd} edition (1989);

D. Chulin, M. Deleuil, Y. Pourcelot, Powder Technology and Pharmaceutical Processes, in J. C. Williams, T. Allen, Handbook of Powder Technology, Elsevier Amsterdam-London New York-Tokyo, (1994);

J. T. Carstensen, Pharmaceutical Principles of Solid Dosage Forms, Technomic Publishing Co., Inc., Lancaster-Basel, (1993).

### PRODUCTION EXAMPLE

For the production of effervescent formulations, 50, 100, 300, 400, 500 or 600 mg N-acetylcysteine per dosage unit or the corresponding amount of glutathione; 2, 5, 10, 20, 25, 30 or 50 to 60 mg of (a) the ascorbic acid compound; of (c) one or more carotinoids; of (c) one or more tocopherols and/or of (d) one or more flavonoids such as rutin or esculine, and optionally polyphenols such as caffeic acid (amide) or suitable extracts from citrus fruits or green tea were processed in a customary manner with pharmaceutically acceptable adjuvants to a granulate or powder at the dose of the individual dosages named, wherein, aside from carbonates such as preferably monsodium carbonate, organic acids such as tartaric acid and/or citric acid were preferably used as carbon dioxide developing substances and the final mixture was compacted if required under optional addition of intestinal lubricants, anti-sticking agents and lubricating agents as well as further customary tableting adjuvants if required. In the case of the combination with NSAID, especially with Diclofenac, an amount of 10, 25, 50, 100, 200, 300, 400, 500 or 600 or more mg per dosage unit was preferably processed to a separate granulate or powder depending on the desired strength of activity, activity equivalent and type of activity and/or pharmakinetics, added to the above prepared granulate and compressed to tablets in the customary manner if desired. The given amounts of active ingredient(s) and stabilizer mixture can be respectively halved or also doubled or trebled if required depending on the dosage. The medicinals obtained in this manner are manufactured in the customary manner protected from moisture.

1. Stabilized medicament with an amount of cysteinyl or dicysteinyl compounds, particularly N-acetylcysteine, against broncial and pulmonary diseases, and/or glutathione, optionally in combination with a non-steroid antiphlogistic agent/ analgesic agent (NSAID), especially Diclofenac, for suppressing organ, especially liver, damage and inflammation damage as sole active ingredient(s), containing a stabilizer mixture comprising at least 3 components from the series
a) ascorbic acid (vitamin C) or salts or esters thereof,
b) one or more tocopherols (vitamin E),
c) one or more carotinoids and/or vitamin A,
d) one or more natural or synthetic flavonoids, bioflavonoids, flavanols or catechins including anthocyanenes and glycosides thereof
as well as pharmaceutically acceptable additives and/or carrier agents for per oral, topical, parenteral or rectal administration forms if required.

2. Stabilized medicament according to claim 1, characterized in that the non-steroid antiphlogistic agent is selected from the group consisting of paraaminophenols, particularly paracetamol, salicylates, particularly acetylsalicylic acid, diflunisal or choline salicylate, acetic acid derivatives such as indometacin or acemetacin, propionic acid derivatives, particularly ibuprofen, ketoprofen or naproxen, indole derivatives, particularly sulindac, oxicam derivatives such as priroxicam, fenamate derivatives such as mefenaminic acid or pyrzole derivatives, particularly phenylbutazone, phenazone, propylphenazone or metamizole, as well as their optionally existing pharmacologically active enantomers or other optical isomers.

3. Stabilized medicament according to claim 1 or 2, characterized in that the component b) of the stabilizer mixture is selected from one or more compounds of natural or synthetic origin from the series α-, β-, γ-, δ- and ε-tocopherol as well as all rac-α-(-tocopherol, tocol, α-tocopherolhydroquinone, α-tocopherolquinone, or derivatives thereof such as acetates, succinates, ubiquinones, boviquinones, plastoquinones or menaquinones.

4. Stabilized medicament according to the claims 1 to 3, characterized in that the component c) of the stabilizer mixture is selected from one or more compounds, as well as their derivatives of natural or synthetic origin, from the series cathaxanthin, rhodoxanthin, capsorubin, zeaxanthin, α-, β-, γ-, δ-and/or ζ-carotine, lycopin, capsanthin, cryptoxanthin, crocetin, lutein, decapren-β-carotine, dodecapren-β-carotine, astaxanthin, violaxanthin or bixin.

5. Stabilized medicament according to the claims 1 to 4, characterized in that the component d) is selected from one or more compounds as well as their derivatives of natural or synthetic origin, such as glucosides, from the series of flavonoids and/or bioflavonoids or flavinols such as chrysin, apigenin, fisetin, kaempferol, luteolin, galangin, gossypetin, morin, myricetin, naringin, quercetin, robinetin, anthocyanins, rutin, hesperidine, taxifolin, catechinic acid, epicatechol, epicatechol gallate, gallocatechol, epigallocatechol gallate, tangeretin, eriodictyol, naringenin, rutin, troxerutin, (quercetinrutin), esculin, esculetin, skimmin, umbelliferon, corresponding other glycosides such as esculoside or anthocyanosides, rutosides such as tri(hydroxyethyl)rutoside, ruscogenins, O-(β-Phydroxyethyl)-rutocides as well as natural extracts from citrus fruits, Myrtillum species, Melilotus species, Hypericum species, or other plant extracts supplying bioflavonoids.

6. Stabilized medicament according to the claims 1 to 5, characterized in that one or more compounds of natural or synthetic origin from the series of polyphenols such as caffeic acid, caffeic acid amide, ethoxyquin, carosinic acid, carnosol, as well as derivatives of these compounds, extracts from thea species, rosemary species or other plants supplying natural phenol compounds of these type, are contained as additional stabilizing agents with respect to component d).

7. Stabilized medicament according to the claims 1 to 6, characterized in that the liquid aqueous or lipid containing formulations contain as solubilizers a solubility promoter from the series polyoxyethylene glycol fatty acid esters in the form of monolaurate, monostearate, monooleate, triricinoleate or trihydroxystearate (cremophor types, tegin types, phospholipids such as for example, lecithins, aside from optional organic, pharmaceutically acceptable solvents such as alcohols in the from of ethanol, isopropanol, butanol, wherein one or more active ingredients can be present, individually or in combination with each other, particularly N-acetylcysteine or one or more non-steroid inflammation inhibitors (antiphlogistic agents/ analgesics, in liposomal, finely dispersed or target form or also micronized.

8. Stabilized medicament according to the claims 1 to 7, characterized in that the quantitative relation of (i) cysteinyl and/or dicysteinyl derivatives, particularly N-acetylcysteine, and/or (ii) glutathione : (iii) one or more non-steroid antiphlogistic agents (NSAID) as well as: (iv) the stabilizer mixture is 0-2.0 percent by weight (i) and/or 0-2.0 percent by weight (ii): 0-2.0 percent by weight (iii): 0.05-1.0 percent by weight (iv).

9. Medicament according to claims 1 to 8, characterized in that it is present in the form of optionally coated, gastric juice resistant and/or retarded release capsules, compressives, sublingual or chewable tablets, chewing gum, lozenges, effervescent formulations, tabs, granulates, pellets, microcapsules, dry juice, powders, injection and infusion preparations, suppositories, drops, suspensions, solutions, dosing aerosols, nebulizers, atomizers, sprays, liposome formulations, transdermal formulations, plasters, pastes, emulsions, creams of gels.

10. Stabilized medicament according to the claims 1 to 8, characterized in that in each case one or more active ingredients from the group of the respective stabilized cysteinyl-containing compound(s), and optionally glutathione as well as the NSAID compound(s), are present side by side in the medicament package in separate form in the medicinal formulation and/or as separate medicines, for example as per oral solid medicines, particularly in the form of compressives or capsules on the one hand and vials on the other hand

11. Stabilized medicament according to the claims 1 to 9, characterized in that it is present in the form of an effervescent tablet, effervescent granulate or tabs, wherein the active ingredient or active ingredients are present (i) as cysteinyl compounds in individual dosages of 10 mg, 20 mg, 25 mg, 50 mg, 100 mg or 200 mg or the respective 2-, 3-, 4- or 10-fold amount thereof, particularly as N-acetylcysteine, together with the stabilizer mixture of at least 3 or 4 components a), b), c) and d) from the series ascorbic acid compound, vitamin A (carotinoids), vitamin E (tocopherols) and/or one or more flavonoids such as rutin, esculin, catechinic acid, plant polyphenols such as caffeic acid and/or extract(s) from citrus fruits containing bioflavonoids in individual dosages of 2.0 mg, 2.5 mg, 5.0 mg, 10 mg or 20 mg each and/or the respective 2-, 3-, 4-, 5- or 10-fold amount thereof, optionally in combination with glutathione (ii) in individual dosages of 25 mg, 50 mg, 100 mg, 200 mg or 250 mg and/or the respective 2-, 3-, or 4-fold amount thereof and optionally further in combination with (iii) NSAID compounds, particularly Diclofenac, each in an amount of 25 mg, 50 mg, 100 mg or 200 mg and/or the respective 2-, 3-, 4-, 5- or 10-fold amount thereof together with customary tableting and granulating adjuvants, in addition to organic acids developing carbon dioxide in effervescent formulations such as citric or tartaric acid and carbonates such as monosodium carbonate, granulation adjuvants, flavoring agents, sweetening agents such as saccharin and/or sugars such as mannitol, sorbitol and customary carrier agents if necessary.

12. Stabilized medicament according to the claims 1 to 11, characterized in that it additionally contains trace elements, particularly selenium, for example in an individual or daily dose amount of, in each case, 0.01, 0.02, 0.05, 0.1, 0.2, 0.24, 0.4, 0.5 up to a maximum 0.8 or 1.0 mg as selenite or selenate, for example as the sodium salt.

13. Medicament according to the claims 1 to 12, characterized in that is entirely free of the stabilizer mixture a), b), c) and/or d) and/or merely contains one, two or three of these stabilizer components.

14. Use of cysteinyl or dicysteinyl compounds, particularly N-acetylcysteine and/or glutathione together with a stabilizer mixture comprising at least 3 components from the series
a) ascorbic acid (vitamin C) or salts or esters thereof,
b) one or more tocopherols (vitamin E),
c) one or more carotinoids and/or vitamin A,
d) one or more natural or synthetic flavonoids, bioflavonoids, flavanols or catechins including anthocyanenes and glycosides thereof
as well as pharmaceutically acceptable additives and/or carrier agents for per oral, topical, parenteral or rectal administration forms if required, optionally in combination with a non-steroid antiphlogistic agent/analgesic agent (NSAID), especially Diclofenac, as sole active ingredient(s) for the production of medicaments for the therapeutic or prophylactic treatment of inflammatory diseases of the respiratory tract and for suppression of organ and inflammation damage, particularly of the liver.

15. Use according to claim 14 for the production of medicaments for the treatment of inflammatory diseases of the bronchia and lungs such as otitis, sinusitis, laryngitis, mucovisidosis, rheumatic disorders and/or inflammation, states of pain, radiation damage, for detoxification, especially of medicinal damage, for the prevention and therapy of carcinomas, optionally in combination with cytostatic agents, for organ perfusion or for the prevention of transplantation rejection.

16. Use of a mixture, comprising at least 3 components of the series
a) ascorbic acid (vitamin C) or salts or esters thereof,
b) one or more tocopherols (vitamin E),
c) one or more carotinoids and/or vitamin A,
d) one or more natural or synthetic flavonoids, bioflavonoids, flavanols or catechins including anthocyanenes and glycosides thereof
for the stabilization of medicines with an amount of one or more cysteinyl compounds, particularly N-acetylcysteine, as well as pharmaceutically acceptable additives and/or carrier agents for per oral, topical, parenteral or rectal administration forms if required, optionally in combination with one or more NSAID compounds, especially Diclofenac.
